(19) United States
(12) Patent Application Publication
(10) Pub. No.: US 2013/0022559 A1
(43) Pub. Date: Jan. 24, 2013

(54) Methods For Inhibiting Tyrosinase Using an Extract of Laminaria Saccharina

(71) Applicants: Cheri Lynn Swanson, West Chester, OH (US); Tomohiro Hakozaki, Cincinnati, OH (US); Leo Timothy Laughlin, II, Mason, OH (US)

(72) Inventors: Cheri Lynn Swanson, West Chester, OH (US); Tomohiro Hakozaki, Cincinnati, OH (US); Leo Timothy Laughlin, II, Mason, OH (US)

(21) Appl. No.: 13/189,182

(22) Filed: Jul. 22, 2011

(51) Int. Cl. A61K 36/03; A61K 8/99; A61P 29/00; A61Q 19/02; A61Q 17/04; A61K 8/67; A61Q 1/02

(52) U.S. Cl. 424/59; 424/195.17; 424/63

(57) ABSTRACT

A method of inhibiting tyrosinase activity may involve the step of applying a composition comprising a Laminaria Saccharina extract to a substrate in need of tyrosinase inhibition. The method may further comprise a step of identifying a substrate in need of tyrosinase inhibition. The composition may be left on the substrate and/or repeatedly applied to the substrate to achieve the desired inhibition of tyrosinase activity.

### FIELD OF THE INVENTION

The present invention relates to methods for inhibiting tyrosinase activity by using an extract of Laminaria Saccharina.

### BACKGROUND OF THE INVENTION

Melanin is produced by a complex set of reactions within the melanocyte involving, at a basic level, the enzyme tyrosinase and the protein L-tyrosine. It is well recognized that tyrosinase is an essential component of melanin synthesis. Tyrosinase catalyzes the conversion of L-tyrosine to DOPA (L-3,4-dihydroxyphenylalanine) and of DOPA to dopaquinone. Dopaquinone undergoes further conversion to form melanin. A need exists for novel methods and compositions by which to inhibit tyrosinase activity.

Extracts of Laminaria Saccharina, a species of brown algae, are known in the art. One example is sold under the tradename Phlorogine by Biotech Marine, France. Phlorogine Phlorogine is known as anti-seborrhoeic agent that can regulate the activity of sebaceous glands, as described for example in United States Patent Application Publication No. 2008/0119527A1. Extraction methods for brown algae are also known. European Patent No. 1074262B 1 describes an extraction method for the class Phaeophyceae and the species Laminaria Ochroleuca. These extracts are described as being used in cosmetic compositions as an osmoprotector, free-radical scavenger, or against the effects of skin aging effects. A cosmetic composition sold under the brand name SK-II Facial Clear Solution (Procter & Gamble, Cincinnati, Ohio) has a concentration of Phlorogine of about 1.25%. The SK-II Facial Clear Solution is marketed as a gel hydrator that moisturizes the skin without increasing oily shine.

### SUMMARY OF THE INVENTION

A method of inhibiting tyrosinase activity comprising the step of applying a composition comprising a Laminaria Saccharina extract to a substrate in need of tyrosinase inhibition.

A method of inhibiting tyrosinase activity of a skin surface, the method comprising the steps of identifying a substrate in need of tyrosinase inhibition, wherein the substrate is an area on the skin surface; and applying a composition comprising a Laminaria Saccharina extract to the area, wherein the composition comprises 0.025% or less of a Laminaria Saccharina extract.

A method of inhibiting tyrosinase activity of a facial skin surface, the method comprising the steps of selecting an area on the facial skin surface in need of tyrosinase inhibition, wherein the area is a hyperpigmented spot, applying a composition comprising a Laminaria Saccharina extract to the area, wherein the composition comprises 0.025% or less of a Laminaria Saccharina extract, and leaving the composition on the area for at least about 1 hour.

In response to the technical problems identified in the background, the present invention may take other forms. Further forms of the present invention will be appreciated from the detailed description that follows.

### DETAILED DESCRIPTION OF THE INVENTION

All percentages and ratios used herein are by weight of the total composition and all measurements made are at 25° C., unless otherwise designated. All numeric ranges are inclusive of narrower ranges; delineated upper and lower range limits are interchangeable to create further ranges not explicitly disclosed.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “apply” or “application”, as used in reference to a composition, means to place a composition of the present invention into contact with a suitable substrate.

The term “dermatologically acceptable,” as used herein, means that the compositions or components described are suitable for use in contact with human skin tissue without undue toxicity, incompatibility, instability, allergic response, and the like.

The term “safe and effective amount” as used herein means an amount of a compound or composition sufficient to significantly induce a positive benefit.

The term “post-inflammatory hyperpigmentation” as used herein refers to an acute or temporary increase in pigmentation as a response to a transient inflammatory event, especially in dark skin subjects. Post-inflammatory hyperpigmentation typically subsides once the transient inflammatory event dissipates. Examples of transient inflammatory events include, but are not limited to, acne lesions, ingrown hairs, scratches, insect bites, surfactant damage, and short-term UV exposure.

The term “hyperpigmented spot” as used herein refers to a defined area of skin wherein the pigmentation is greater than that of an adjacent area of skin due to localized and chronic or systemic overproduction of melanin. Hyperpigmented spots can include one or more of age spots, sun spots, solar lentigos, hypo-melanotic lesions, freckles, and melasma spots.

The term “age spots” as used herein refers to a defined area of skin wherein the pigmentation is greater than that of adjacent skin due to localized and chronic overproduction of melanin caused by intrinsic or extrinsic aging factors.

The term “skin tone agent” as used herein refers to an agent that regulates melanin production signals, synthesis of melanin, systemic transfer of melanin between the melanocyte and the keratinocyte, and/or melanin degradation. Skin tone agents can improve the appearance of uneven skin tone by acting as a lightening or pigmentation reduction cosmetic agent. Skin tone agents can be identified using biochemical assays, cell based assays, skin based ex-vivo assays, and/or en vivo testing.

The term “skin tone” as used herein refers to the overall appearance of melanin in the skin caused by the systemic, rather than transient, synthesis of melanin. Skin tone is typically characterized over a larger area of the skin. The area ideally may be than 100 mm², but larger areas are envisioned such as the entirety of the facial skin or any of the facial skin surfaces. Skin tone can be measured by image analysis. For example, overall lightness can be measured by L* coordinate in L*a*b* color space (International Commission on Illumination). Chromophore mapping such as melanin mapping may be used as an indicator of overall skin tone. Mean melanin may be calculated from the chromophore map data. Additionally, skin ton evenness can be determined by melanin evenness which also may be determined calculated from the chromophore map data. Suitable chromophore mapping techniques are discussed in the example below.

The term “facial skin surfaces” as used herein refers to one or more of forehead, periorbital, cheek, perioral, chin, and nose skin surfaces.

### I. Laminaria Saccharina Extract

Compositions of the present invention include a safe and effective amount of Laminaria Saccharina extract, a brown algae extract. The compositions of the present invention may comprise Laminaria Saccharina extract in any amount allowing for reasonable delivery of the composition to a substrate. Surprisingly, it has been found that small quantities of Laminaria Saccharina extract provide appreciable tyrosinase inhibition effect. The compositions of the present invention may comprise Laminaria Saccharina extract in amounts less than about 1.25%, 0.5%, 0.25%, 0.125%, 0.075%, 0.0250%, 0.0125%, 0.0063%, or 0.0031%. The compositions of the present invention may comprise Laminaria Saccharina extract in amounts greater than about 0.00125%, 0.0025%, 0.005%, or 0.01%. The delineated upper and lower range limits are interchangeable to create ranges not explicitly disclosed. The Laminaria Saccharina extract can be prepared by processes known in the art, such as, for example, described in European Patent No. 1074262B1.

A suitable Laminaria Saccharina extract containing composition is commercially available as Phlorogine and/or Phlorogine BG, from Marine Biotech, France. Phlorogine and/or Phlorogine BG contain approximately about 1% to about 2.5% dry Laminaria Saccharina extract with the remaining material being inert carrier. Another suitable Laminaria Saccharina extract is available via product code HG 657 from Ennagram, France. Other suitable compositions may be formed by combining Laminaria Saccharina extract (such as Phlorogine or Phlorogine BG) with additional materials. The Laminaria Saccharina extract containing composition may further comprise a dermatologically acceptable carrier, a tone agent, an anti-inflammatory, a sunscreen active, and/or other actives and agents as described below.

### II. Optional Ingredients

A. Dermatologically Acceptable Carrier

The compositions of the present invention may also comprise a dermatologically acceptable carrier (“carrier”) for the composition. The phrase “dermatologically acceptable carrier”, as used herein, means that the carrier is suitable for topical application to the keratinous tissue, has good aesthetic properties, is compatible with the actives of the present and will not cause any safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. Non-limiting examples include simple solutions (aqueous or oil based), emulsions, and solid forms (gels, sticks, flowable solids, amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

The aqueous phase typically comprises water. However, in other embodiments, the aqueous phase may comprise components other than water (non-water components), including but not limited to water-soluble moisturizing agents, conditioning agents, anti-microbials, humectants and/or other water-soluble skin care actives. In one embodiment, the non-water component of the composition comprises a humectant such as glycerin and/or other polyols. However, it should be recognized that the composition may be substantially (i.e., less than 1% water) or fully anhydrous.

A suitable carrier is selected to yield a desired product form. Furthermore, the solubility or dispersibility of the compositions components (e.g., Laminaria Saccharina extract, sunscreen active, additional components) may dictate the form and composition of the carrier. In one embodiment, oil-in-water or water-in-oil emulsions are preferred.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

B. Skin Tone Agent

In some embodiments, it may be desirable to include a skin tone agent in the composition in combination with the Laminaria Saccharina extract. The skin tone agent may be included to further improve overall skin tone. When present, the compositions of the present invention contain up to about 50%, 40%, 30%, 20%. 10%. 5%, or 3%, by weight of the composition, of the skin tone agent. When present, the compositions of the present invention contain at least about 0.001%, 0.01%, 0.1%, 0.2%, 0.5%, or 1%, by weight of the composition, of the skin tone agent. Suitable ranges include any combination of the lower and upper limits including suitable ranges from about 0.1% to about 50%; from about 0.2% to about 20%; or from about 1% to about 10%, by weight of the composition, of the skin tone agent. The amounts listed herein are only to be used as a guide, as the optimum amount of the skin tone agent will depend on the specific active selected since their potency does vary considerably.

Suitable skin tone agents include, but are not limited to, sugar amines, vitamin B3 compounds, arbutin, deoxyarbutin, sucrose dilaurante, bakuchoil (4-[(1E, 3S)-3-ethenyl-3,7-dimethyl-1,6 octadienyl]phenol or monterpene phenol), pyrenoine (available from Biotech Marine, France), panicum miliaceum seed extract, arlatone dioic acid, cinnamic acid, ferulic acid, achromaxyl, methyl nicotinamide, oil soluble licorice extract, folic acid, undecylenic acid (i.e., undecenoic acid), zinc undecylenate, thiamine (Vitamin B1) and its hydrochloride, L-tryptophan, hexylrescorcinol, helianthus annuus (sunflower) and vitis vinifera (grape) leaf extract, carnosine (i.e., dragosine), methyl gentisate, 1,2-hexandiol and 1,2-octandiol (i.e., combination sold as Symdiol 68 by Symrise AG, Germany), inositol, decylenoylphenylalanine (e.g., sold under the tradename Sepiwhite by Seppic, France), koijic acid, hexamidine compounds, salicylic acid, and retinoids including retinol and retinyl propionate.

In certain embodiments, the skin tone agent is selected from vitamin B3 compounds, sugar amines, hexamidine compounds, salicylic acid, and retinoids. As used herein, “vitamin B₃ compound” means a compound having the formula:

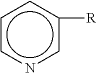

wherein R is —CONH₂ (i.e., niacinamide), —COOH (i.e., nicotinic acid) or —CH₂OH (i.e., nicotinyl alcohol); derivatives thereof; and salts of any of the foregoing. As used herein, “sugar amine” includes isomers and tautomers of such and its salts (e.g., HCl salt) and its derivatives. Examples of sugar amines include glucosamine, N-acetyl glucosamine, mannosamine, N-acetyl mannosamine, galactosamine, N-acetyl galactosamine, their isomers (e.g., stereoisomers), and their salts (e.g., HCl salt). As used herein, “hexaminide compound” means a compound having the formula:

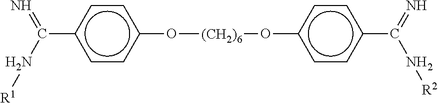

wherein R¹ and R² are optional or are organic acids (e.g., sulfonic acids, etc.). In one embodiment, hexamidine compound includes hexamidine diisethionate.

C. Anti-Inflammatory Agents

Hyperpigmentation may result from skin inflammation. Transient inflammatory events triggering hyperpigmentation and, more specifically, post-inflammatory hyperpigmentation include, but are not limited to, acne lesions, ingrown hairs, scratches, insect bites, surfactant damage, and short-term UV exposure. Inflammation induced hyperpigmentation including post-inflammatory hyperpigmentation may be managed by incorporating into the compositions of the present invention an anti-inflammatory agent. When present, the compositions of the present invention contain up to about 20%. 10%. 5%, 3%, or 1% by weight of the composition, of the anti-inflammatory agent. When present, the compositions of the present invention contain at least about 0.001%, 0.01%, 0.1%, 0.2%, 0.3% 0.5%, or 1%, by weight of the composition, of the anti-inflammatory agent. Suitable ranges include any combination of the lower and upper limits. Suitable anti-inflammatory agents include, but are not limited to nonsteroidal anti-inflammatory agents (NSAIDS including but not limited to ibuprofen, naproxen, flufenamic acid, etofenamate, aspirin, mefenamic acid, meclofenamic acid, piroxicam and felbinac), glycyrrhizic acid (also known as glycyrrhizin, glycyrrhixinic acid, and glycyrrhetinic acid glycoside) and salts such as dipotassium glycyrrhizate, glycyrrhetenic acid, licorice extracts, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, red clover extract, and sea whip extract, derivatives of any of the foregoing, and mixtures thereof.

D. Sunscreen Actives

The compositions of the subject invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in Personal Care Product Council's International Cosmetic Ingredient Dictionary and Handbook, Thirteenth Edition, as “sunscreen agents.” Particularly suitable sunscreen actives are 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

In one embodiment, the composition may comprise from about 1% to about 20%, and alternatively from about 2% to about 10% by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

E. Other Actives and Agents

The compositions of the present invention may contain a variety of other ingredients that are conventionally used in given product types provided that they do not unacceptably alter the benefits of the invention. When present, compositions of the present invention may contain from about 0.0001% to about 50%; from about 0.001% to about 20%; or, alternately, from about 0.01% to about 10%, by weight of the composition, of the other actives and agents. The amounts listed herein are only to be used as a guide, as the optimum amount of the optional components used in a composition will depend on the specific active selected since their potency does vary considerably. Hence, the amount of some actives and agents useful in the present invention may be outside the ranges listed herein.

The optional actives and agents, when incorporated into the composition, should be suitable for use in contact with human skin tissue without undue toxicity, incompatibility, instability, allergic response, and the like within the scope of sound judgment. The compositions of the present invention may include optional actives and agents such as anti-acne actives, desquamation actives, anti-cellulite agents, chelating agents, flavonoids, tanning active, non-vitamin antioxidants and radical scavengers, hair growth regulators, anti-wrinkle actives, anti-atrophy actives, minerals, phytosterols and/or plant hormones, N-acyl amino acid compounds, antimicrobial or antifungal actives, and other useful skin care actives, which are described in further detail in U.S. application publication No. US2006/0275237A1 and US2004/0175347A1.

The Personal Care Product Council's International Cosmetic Ingredient Dictionary and Handbook, Thirteenth Edition, describes a wide variety of non-limiting cosmetic and pharmaceutical ingredients commonly used in the skin care industry, which are suitable optional components for use in the compositions of the present invention. Examples of these ingredient classes include: abrasives, absorbents, aesthetic components such as fragrances, pigments, colorings/colorants, essential oils, anti-caking agents, antifoaming agents, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, and thickeners.

### III. Exemplary Compositions

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minor materials will vary depending on the physical and chemical characteristics of the particular ingredients selected to make the present invention as described herein.

All Examples may be used to inhibit tyrosinase activity. The Examples may suitable for application to a substrate in need of tyrosinase inhibition. The Examples are believed to be particularly suitable for application to a skin surface in need of tyrosinase inhibition.

| Component | Ex. A | Ex. B | Ex. C | Ex. D | Ex. E | Ex. F |
|---|---|---|---|---|---|---|
| Phlorogine or | 1.000 | 1.000 | 1.000 | 1.000 | 1.000 | 2.000 |
| Phlorogine BG *1 | | | | | | |
| N-Acetylglucosamine | 0 | 0 | 2.000 | 0 | 0 | 0 |
| Hexamidine | 0 | 0 | 0 | 0.090 | 0.090 | 0 |
| Diisethionate | | | | | | |
| Undecylenoyl- | 0 | 1.000 | 0.500 | 0 | 0 | 0 |
| phenylalanine *2 | | | | | | |
| (neutralized) | | | | | | |
| Dipotassium | 0 | 0.300 | 0.100 | 0.100 | 0.100 | 0 |
| Glycyrrhizate | | | | | | |
| Niacinamide | 0 | 5.000 | 5.000 | 5.000 | 5.000 | 5.000 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Cetearyl glucoside + | 0.200 | 0.200 | 0.200 | 0.200 | 0.200 | 0.200 |
| cetearyl alcohol *3 | | | | | | |
| Behenyl alcohol | 0.400 | 0.400 | 0.400 | 0.400 | 0.400 | 0.400 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Polyacrylamide + C13-14 | 2.000 | 2.000 | 2.000 | 2.000 | 2.000 | 2.000 |
| isoparaffin + laureth-7 *4 | | | | | | |
| Disodium EDTA | 0.100 | 0.100 | 0.100 | 0.100 | 0.100 | 0.100 |
| Benzyl alcohol | 0.400 | 0.400 | 0.400 | 0.400 | 0.400 | 0.400 |
| Dimethicone + | 2.000 | 2.000 | 2.000 | 2.000 | 2.000 | 2.000 |
| dimethiconol *5 | | | | | | |
| Homosalate | 0 | 0 | 0 | 0 | 9.000 | 0 |
| Avobenzone | 0 | 0 | 0 | 0 | 3.000 | 0 |
| Octocrylene | 0 | 0 | 0 | 0 | 2.600 | 0 |
| Oxybenzone | 0 | 0 | 0 | 0 | 1.000 | 0 |
| Octisalate | 0 | 0 | 0 | 0 | 4.500 | 0 |
| Water | QS | QS | QS | QS | QS | QS |
| TOTAL | 100 | 100 | 100 | 100 | 100 | 100 |

*1 Available from Biotech Marine, France.
*2 Sepiwhite available from SEPPIC, France.
*3 Emulgade PL 68/50 available from Cognis GmbH.
*4 Sepigel 305, available from SEPPIC, France.
*5 Dow Corning DC1503 available from Dow Corning, Inc., Midland, MI.

The compositions of the present invention are generally prepared by conventional methods such as are known in the art of making compositions and topical compositions. Such methods typically involve mixing of the ingredients in one or more steps to a relatively uniform state, with or without heating, cooling, application of vacuum, and the like. Typically, emulsions are prepared by first mixing the aqueous phase materials separately from the fatty phase materials and then combining the two phases as appropriate to yield the desired continuous phase. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### IV. Methods

Methods of inhibiting tyrosinase activity may involve application of the aforementioned composition. The composition may be applied to a substrate in need of tyrosinase inhibition. Suitable substrates in need of tyrosinase inhibition may be selected or indentified as part of the method. For example, the substrate in need of tyrosinase inhibition includes any substrate containing tyrosinase which an individual selects for inhibition. The substrate may be a simple solution such as the phosphate buffered saline solution used in the Tyrosinase Inhibition Assay described below. In one embodiment, the substrate may be a plant or animal tissue. In certain embodiments, the substrate is a skin surface. A suitable skin surfaces include facial skin surfaces, hand and arm skin surfaces, foot and leg skin surfaces, and neck and chest skin surfaces (e.g., décolletage). In certain embodiments, a particular area or areas of the skin surface may be selected for tyrosinase inhibition. In one embodiment, the area may be the facial skin surface including the forehead, perioral, chin, periorbital, nose, and/or cheek.

In another embodiment, the area on the skin surface is a hyperpigmented spot or other area with increased melanin production. The hyperpigmented spot may be identified by the user or a third party such as a dermatologist, cosmetician, or other caregiver. Identification may be done by visual inspection of the skin for hyperpigmented spots in need of treatment based on size and/or color. Identification may also be done by commercially available imaging devices such SlAscope® V (available from Astron Clinica, Ltd., UK) or the VISIA® Complexion Analysis system (available from Canfield Scientific, Inc., Fairfield, N.J.). Both devices are capable of collecting images of the skin and identifying hyperpigmented spots. In some instances, the method comprises the step of identifying a plurality of hyperpigmented spots.

The composition may be applied and left on the substrate for a sufficient contact time and/or repeatedly applied a sufficient number of times to achieve the desired inhibition of tyrosinase. In certain embodiments, the contact time is greater than about 1 hour, 2 hours, 6 hours, 8 hours, 12 hours, or 24 hours. The contact time is time from application of the composition until the composition is removed. In certain embodiments, the composition may be removed by rinsing or washing the substrate. When a skin surface is selected as the substrate, the composition may be removed by washing or rinsing the skin. The treatment period may involve a single application or multiple applications. The composition may be applied at least daily. In other embodiments, the composition is applied at least twice daily. Multiple applications may occur over the course of at least about 1 week. Alternately, the treatment period may last more than about 4 weeks or more than about 8 weeks. In certain embodiments, the treatment period will extend over multiple months (i.e., 3-12 months) or multiple years.

### V. Experimental Examples—Tyrosinase Inhibition Assay

The following experimental example is provided to illustrate certain features and advantages of various embodiments of the invention and should not be construed as limiting the scope thereof.

This assay can identify agents that may interfere with the ability of mushroom tyrosinase enzyme to convert L-tyrosine to L-dihydroxyphenylalanine (L-DOPA). Mushroom Tyrosinase, available from Sigma-Aldrich, Missouri, USA (item T3824), is employed in the assay. The substrate solution may include a 1× concentrated phosphate buffered saline (PBS) (pH 7.4), available from Invitrogen, California, USA (GIBCO catalogue number 10010-023). A positive control may be employed utilizing 4-Hydroxyphenyl-β-D-glucopyranoside (Arbutin), available from Sigma-Aldrich, Missouri, USA.

The assay also uses dimethyl sulfoxide (DSMO), available from Sigma-Aldrich, Missouri, USA, (item D5879), and Falcon® 1172 Microtest™ non-tissue culture treated, clear, flat bottom 96 well plates. Tyrosinase Inhibitor is determined by a UV-Visible Spectrum Plate Reader, such as a SpectraMax250, available from Molecular Devices, California, USA, coupled with data acquisition and analysis software such as SoftMax Pro, available from Molecular Devices, California, USA. The assay steps include:

### I. Prepare Reagents and Positive Controls

a. 1 mM Enzyme substrate working solution —Add 0.01812 g L-tyrosine to 100 mL 1×PBS. Sonicate until L-tyrosine is dissolved. Vortex as necessary. Store at 4° C. when not in use
b. 20 mM Positive Control—A 0.2M stock solution of Arbutin positive control is prepared by adding 0.0544 g Arbutin to 1 mL DMSO. Vortex and sonicate for 1 minute until Arbutin is dissolved. Dilute this solution 1:10 by adding 100 uL to 900 uL DMSO for a working solution of 20 mM Arbutin. Store at room temperature until used.
c. Phlorogine Test Compound—Phlorogine is diluted in sufficient water to yield needed concentrations. Final volume of test compound in the assay is 2 μL, so working solutions are typically made up at 5-40 mM (100×), which yields a final concentration of 50-400 μM in the assay.
d. Tyrosinase Enzyme—Reconstitute tyrosinase enzyme at 1000 U/mL with cold 1×PBS buffer. Store this stock solution in 1 mL aliquots protected from light at −20° C. until needed. Enzyme working solution of 26 U/mL is prepared by adding 1 mL thawed stock solution (1000 U/ml) to 37.5 mL cold 1×PBS buffer. This is enough enzyme for four 96-well plates. Protect from light and keep on ice until used in the assay.

### II. Assay Methodology

1. Add 200 uL 1×PBS buffer to triplicate wells on each test plate for proper blank.
2. Add 2 uL DMSO to triplicate wells for a vehicle control.
3. Add 2 uL Arbutin to triplicate wells for a positive control.
4. Add 2 uL of the Phlorogine Test Compound to triplicate wells.
5. Add 98 uL tyrosinase enzyme working solution to each well except blanks Mix the compounds with the enzyme by pipetting up and down twice or vortex briefly.
6. Add 100 uL/well of L-tyrosine substrate.
7. Choose kinetic setting on the SpectraMax 250 Plate Reader and record absorbance readings at 475 nm every 1 minute for 1 hour.
8. Calculate the slope for the controls and test compounds using the data acquisition software.
9. Calculate the percent inhibition of tyrosinase according to the following formula:

$$\frac{( Avg . vehicle control slope - Avg . sample slope )}{Avg . vehicle control slope} \times 100$$

Using generally the assay outlined above, Phlorogine inhibited tyrosinase activity as shown in Table 1 below.

TABLE 1

| Concentration | Laminaria Saccharina | |
|---|---|---|
| Phlorogine | extract concentration | |
| (w/v %) | (apprx.) | Tyrosinase Inhibition |
| 1% | 0.025%-0.01% | 60% |
| 0.5% | 0.0125%-0.005% | 64% |
| 0.25% | 0.00625%-0.0025% | 73% |
| 0.125% | 0.003125%-0.00125% | 77% |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of inhibiting tyrosinase activity comprising the step of applying a composition comprising a Laminaria Saccharina extract to a substrate in need of tyrosinase inhibition.

2. The method of claim 1, wherein the composition comprises 0.0250% or less of the Laminaria Saccharina extract.

3. The method of claim 1, wherein the composition comprises 0.0125% or less of the Laminaria Saccharina extract.

4. The method of claim 1, wherein the composition comprises 0.0625% or less of the Laminaria Saccharina extract.

5. The method of claim 1, wherein the composition comprises 0.0031% or less of the Laminaria Saccharina extract.

6. The method of claim 1, wherein the method further comprises the step of leaving the composition on the substrate for a contact time sufficient to achieve inhibition of tyrosinase activity.

7. The method of claim 6 wherein the contact time is at least about 1 hour.

8. A method of inhibiting tyrosinase activity of a skin surface, the method comprising the steps of (i) identifying a substrate in need of tyrosinase inhibition, wherein the substrate is an area on the skin surface; and (ii) applying a composition comprising a Laminaria Saccharina extract to the area, wherein the composition comprises 0.025% or less of a Laminaria Saccharina extract.

9. The method of claim 8, wherein the composition comprises 0.0125% or less of the Laminaria Saccharina extract.

10. The method of claim 8, wherein the composition comprises 0.00625% or less of the Laminaria Saccharina extract.

11. The method of claim 8, wherein the composition comprises 0.0031% or less of the Laminaria Saccharina extract.

12. The method of claim 8, wherein the skin surface is a facial skin surface.

13. The method of claim 12, wherein the area on the facial skin surface is a hyperpigmented spot.

14. The method of claim 8, wherein the composition is applied to the area on the skin surface at least once a day for at least about four weeks.

15. The method of claim 8, wherein the composition is applied to the area on the skin surface at least twice a day for at least about four weeks.

16. The method of claim 8, wherein the composition is applied to the area on the skin surface at least once a day for at least about eight weeks.

17. The method of claim 8, wherein the composition is applied to the area on the skin surface at least twice a day for at least about eight weeks.

18. The method of claim 8, wherein the composition further comprises a dermatologically acceptable carrier.

19. The method of claim 18, wherein the composition further comprises a skin tone agent selected from vitamin B3 compounds, arbutin, deoxyarbutin, sucrose dilaurante, bakuchoil, pyrenoine, panicum miliaceum seed extract, arlatone dioic acid, cinnamic acid, ferulic acid, achromaxyl, methyl nicotinamide, oil soluble licorice extract, folic acid, undecylenic acid, zinc undecylenate, thiamine, thiamine hydrochloride, L-tryptophan, hexylrescorcinol, helianthus annuus and vitis vinifera leaf extract, carnosine methyl gentisate, 1,2-hexandiol and 1,2-octandiol, inositol, decylenoylphenylalanine, koijic acid, hexamidine compounds, salicylic acid, retinoids, and combinations thereof.

20. The method of claim 18, wherein the composition comprises a sunscreen active.

21. The method of claim 18, wherein the composition comprises an anti-inflammatory active.

22. The method of claim 21, wherein the anti-inflammatory active is selected from glycyrrhizic acid, glycyrrhizic acid salts, licorice extract, bisabolol, and combinations thereof.

23. The method of claim 8 further comprising a step of leaving the composition on the area on the skin surface for a contact time sufficient to achieve inhibition of tyrosinase activity.

24. The method of claim 23 wherein the contact time is at least about 1 hour.

25. A method of inhibiting tyrosinase activity of a facial skin surface, the method comprising the steps of (i) selecting an area on the facial skin surface in need of tyrosinase inhibition, wherein the area is a hyperpigmented spot, (ii) applying a composition comprising a Laminaria Saccharina extract to the area, wherein the composition comprises 0.025% or less of a Laminaria Saccharina extract, and (iii) leaving the composition on the area for at least about 1 hour.
